(19) United States
(12) Patent Application Publication
(10) Pub. No.: US 2010/0057151 A1
(43) Pub. Date: Mar. 4, 2010

(54) DEVICE FOR THE DEFIBRILLATION OF THE HEART

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/514,382

(22) PCT Filed: Dec. 6, 2007
(86) PCT No.: PCT/EP07/10605

§ 371 Date: May 11, 2009

(30) Foreign Application Priority Data
Feb. 28, 2007 (DE) 102007009716.8

(51) Int. Cl. A61N 1/362; A61N 1/39

(52) U.S. Cl. 607/4

(57) ABSTRACT

A device (1) that serves for the defibrillation of the heart (2), and can be implanted as a whole. The device includes an implantable combined pacemaker and defibrillator (3), at least one defibrillation electrode (6), and a counter electrode (4, 41, 42), and a stimulation and sensor electrode (5) that can also be implanted, wherein the defibrillation electrode (6) can be retracted subcutaneously near the heart exterior in the region of the cardiac apex (2a), such as by a tension element (13) and a needle (12), and can be implanted, and is configured as at least one flexible helix made of metal or biocompatible steel, thus having high flexibility and low space requirement.

### BACKGROUND

The invention relates to a device for the defibrillation of the heart with an implantable, combination cardiac pacemaker and defibrillator, with at least one defibrillation electrode and a counter electrode, as well as with at least one stimulation and sensing electrode that can also be implanted, wherein, in the position of use, the defibrillation electrode is separate from the stimulation electrode and can be implanted subcutaneously close to the outside of the heart in the region of the cardiac apex.

A device of this type is known from WO 82/02664. Here, however, the defibrillation electrode has large dimensions and is large and relatively stiff due to its configuration as a metal mesh with an insulating edge, so that an operation with an open thorax is required.

### SUMMARY

Therefore, the objective arises of creating an implantable device of the type named above in which the thorax does not have to be opened for implanting the defibrillation electrode and the risk of electrode breaking is reduced or ruled out.

For meeting this objective, the device defined above is characterized in that the defibrillation electrode is at least one flexible helix made from metal or biocompatible steel.

Therefore, it is possible to create an access for this flexible defibrillation electrode with relatively small dimensions through a small incision in the skin underneath the ribs, wherein this defibrillation electrode can be, on one hand, incorporated into the tissue adjacent to the heart close to the outside of the heart and can be, on the other hand, connected with its terminal to the implantable cardiac pacemaker and defibrillator in known tunneling technology. A flexible helix can be adapted to the anatomical conditions in the best possible way and can nevertheless output sufficiently large defibrillating current pulses. Here, the advantage is maintained that the defibrillation electrode does not have to be attached to the exterior of the heart, that is, the normal heart movement is not affected.

For the simplest possible implantation, it is useful if tensioning element or thread is provided on the defibrillation electrode constructed as a helix for the subcutaneous implantation of this electrode. The engagement of the tensioning element or thread to the defibrillation electrode is constructed so that the helix forming this defibrillation electrode remains undilated during implantation and when a tensile force acts on the tension element or the thread.

Thus, with the help of such a thread or tension element to which a needle could also be attached in advance, wherein the needle is removed after implantation, the helix is implanted, and placed in the most favorable position relative to the outside of the heart, without being deformed in an undesired way, so that the helix forming the defibrillation electrode remains in its undilated or slightly dilated form also during and after the implantation procedure despite the implantation work with the help of a tension element and a tool or needle attached to this tension element.

The tension element or the thread used for the implantation of the defibrillation electrode can attach at least to the end or end region of the helix at the back in the insertion direction or to a carrier that holds the helix and that absorbs the tensile force during the implantation procedure and the force is kept away from the helix carried or held by this carrier. This represents a useful embodiment of the defibrillation electrode in helix form in which the helix also does not have to be dilated during the implantation.

Here, a favorable embodiment can provide that the tension element or the thread is attached to the end region or end of the carrier at the front in the insertion direction. Thus, the tensile force exerted during implantation is transmitted to the carrier that is implanted, on its side, under tensile force and that here takes along the helix attached to it as well as its feed line.

A modified embodiment can provide that the helix used as a defibrillation electrode is divided in the axial direction into several helix sections that are connected to each other by wires. The helix could also have several sections between which the wire or wires forming it are not twisted, which allows better curving of the helix, especially in the region of the cardiac apex, if anatomical conditions require this configuration.

It is preferred, especially also for an economical production, if the feed line to the helix-shaped defibrillation electrode is an insulated, low-impedance braid or helix.

Thus, it is possible in a simple way that the helix forming the defibrillation electrode is an insulation-stripped projection of the helix-shaped feed line to the defibrillation electrode. Thus, a helix can be easily used both as a feed line and also as a defibrillation electrode, such that the end forming the defibrillation electrode is stripped of insulation or provided without insulation at the front, while, in a simple way, the feed line can be this same helix or multiple helixes with insulation.

Within the wire forming the feed line or helix, a silver matrix or tantalum matrix increasing the electrical conductivity could be provided. In this way, the defibrillation electrode could have a large power output accordingly even for relatively small dimensions.

The carrier holding the defibrillation electrode could have, for example, the length of the electrode or a somewhat greater length than the electrode. In particular, it could project somewhat in the insertion direction, so that the attachment of a tension element to this carrier is easily possible without negatively affecting the helix.

Another configuration of the invention can provide that the defibrillation electrode is formed by at least two helixes that are connected to the feed line by a wire branching point. Therefore, the advantage could also be maintained that the defibrillation electrode has a highly flexible configuration that nevertheless has small dimensions and has an ideal field-strength distribution for the defibrillation, so that only a relatively low shock energy is necessary, wherein, simultaneously, only a minimal subcutaneous surgical intervention is required for the implantation. Simultaneously, the advantage is maintained both for only one helix and also for two helixes, because this defibrillation electrode is completely separated from the stimulation electrode. The high flexibility of the defibrillation electrode and also its feed line leads to good breakage strength and correspondingly long service lives.

The two or more helixes forming the defibrillation electrode could be attached to a common carrier, in particular, running parallel to each other. Thus, the implantation with the help of a tension element and a needle that is attached to this tension element and with which the electrode can be drawn underneath the heart is practically just as easy as the implantation of a defibrillation electrode formed by only one helix, wherein, through the attachment of the tension element or thread to the carrier, an undesired dilation of the helix-shaped defibrillation electrode is also avoided if this electrode has two helixes.

The preferably flat or approximately plate-shaped carrier can be arranged, in the position of use, on the side of the defibrillation electrode facing away from the heart. Therefore, it can simultaneously form shielding for the actual defibrillation electrode on the side facing away from the heart. Accordingly, the shock energy of the defibrillation electrode is directed selectively toward the heart.

Its carrier for the helix-shaped defibrillation electrode or electrodes can be made from insulating material and, as insulating shielding, it can have a larger width than the defibrillation electrode or electrodes themselves and can project laterally past this helix or these helixes forming them—and, as already mentioned, also in the length direction. Therefore, this carrier also acting as shielding can stabilize the position of the defibrillation electrode within the subcutaneous tissue in the position of use.

For example, the width of the carrier acting as insulating shielding can be two-times or three-times or four-times as large as that of the defibrillation electrode, wherein, however, an intermediate value between these dimensions is also possible.

The most favorable dimensional relationship for the helix or helixes forming the defibrillation electrode can provide that the outer diameter of this helix or helixes equals at least five-times, six-times, or seven-times the diameter of the helix-shaped wire or wires or equals an intermediate value. This produces a flexible helix with more favorable outer dimensions that allow a sufficiently large field-strength distribution for the defibrillation and thus a relatively low shock energy.

For successful defibrillation, it is important when the defibrillation electrode and its counter electrodes(s) are placed so that the defibrillation current flows as uniformly as possible through the entire heart. Thus, for the most uniform possible field-strength distribution during the electrical defibrillation, it is important how the counter electrode of the defibrillation electrode is arranged.

The counter electrode for the defibrillation electrode can be formed here as an atrial electrode that can be inserted transvenously into the heart or that can be implanted, in the position of use, outside of the heart. Above all, the second alternative allows the best possible placement of the counter electrode relative to the position of the defibrillation electrode.

It can be favorable when the counter electrode is arranged outside of the heart as a helix in the cardiac pacemaker platform and/or on the feed line of the defibrillation electrode and/or on the feed line of the stimulation electrode, especially on its outer side(s). These feed lines usually run in the thorax above or in the upper side region of the heart, so that, with a defibrillation electrode in the region of the cardiac apex, a good field-strength distribution and a defibrillation current flowing through the entire heart can be achieved.

For the simplest and most economical solution for this configuration of the invention, it can be useful if the feed line to the counter electrode is arranged within the insulation of the defibrillation electrode or the stimulation electrode and extends up to the counter electrode. In this way, the counter electrode arranged usefully as a helix on the outside of the defibrillation electrode or the stimulation electrode and the feed line of this counter electrode can lead to the same plug as the corresponding electrode carrying them and can be easily implanted accordingly. In this case, an atrial electrode does not need to be implanted. Therefore, it can be eliminated.

Above all, for the combination of individual or multiple features and measures described above, an implantable device for the defibrillation of the heart can be produced in which the actual defibrillation electrode can be formed with a space-saving and highly flexible configuration with a long service life due to its helical shape, so that it can be implanted subcutaneously through minimal surgical intervention. In this way, it can be arranged at the most favorable position underneath the heart separated from the stimulation electrode. The implantation is possible in a very simple way with the help of a needle and a tension element, wherein the resulting tensile forces, however, are kept away from the helix itself.

### BRIEF DESCRIPTION OF THE DRAWINGS

Below, embodiments of the invention will be described in greater detail with reference to the drawing. Shown in partially schematized diagrams are:

FIG. 1 is a view of an implantable device according to the invention for defibrillation with a combination cardiac pacemaker and defibrillator arranged in the position of use, an atrial electrode extending from this device and inserted transvenously into the heart, a stimulation and sensing electrode also leading into the heart, and a defibrillation electrode implanted subcutaneously close to the outside of the heart in the region of the cardiac apex shortly after the implantation and still before the separation of the needle used for implantation and attachment to a tension element, wherein the field-strength distribution on the heart is indicated schematically,

FIG. 2 is, at an enlarged scale, a longitudinal section view through the heart and, here, the arrangement of the atrial electrode, the stimulation electrode, and the defibrillation electrode implanted close to the outside in the region of the cardiac apex, wherein the needle used for subcutaneous implantation has not yet been separated from the thread or the tension element,

FIG. 3 is a view of a defibrillation electrode according to the invention formed as a helix made from metal or biocompatible steel with a feed line and tension element to which a curved needle is attached,

FIG. 4 is a view corresponding to FIG. 3, but with a straight-lined needle,

FIG. 5 is a partial view of the defibrillation electrode according to the invention in which parallel helixes are arranged on a common carrier to which the tension element or the thread for the needle is attached,

FIG. 6 is a side view of the helix-shaped defibrillation electrode and the carrier carrying it with the schematized attachment of the tension element or thread on the front end in the implantation direction and the connection to the feed line on the opposite end,

FIG. 7 is a diagram corresponding to FIG. 1, wherein, however, instead of an atrial electrode as a counter electrode to the defibrillation electrode, a helix is provided that is connected via a feed line to the cardiac pacemaker and defibrillator and is located outside of the heart,

FIG. 8 is a view of an arrangement corresponding to FIG. 7 in which, however, the helical counter electrode for the stimulation electrode is arranged on the outside on its feed line, and

FIG. 9 is, at an enlarged scale, a view of the helical counter electrode located on the outside of the feed line of the defibrillation electrode.

### DETAILED DESCRIPTION OF THE PREFERRED EMBODIMENTS

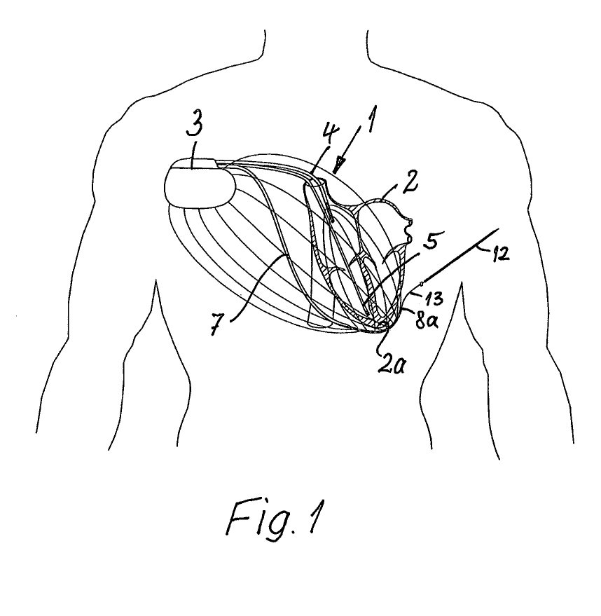

A device designated in FIG. 1 as a whole with 1 is used for the defibrillation of a heart 2 and includes an implantable combination cardiac pacemaker and defibrillator 3, an atrial electrode 4 that can be inserted transvenously into the heart, an implantable stimulation and sensing electrode 5, and a defibrillation electrode that is designated as a whole with 6 and that is connected, in the position of use, like the other electrodes, via a feed line 7 to the defibrillator 3 and that is implanted subcutaneously close to the outside in the region of the cardiac apex 2a. Here, in FIG. 1 the field-strength distribution or the current flow is shown for the case of a defibrillator process that surrounds and encompasses the heart on all sides as much as possible.

Here, with reference to the field lines indicated schematically, one sees that the atrial electrode 4 forms a counter electrode to the defibrillation electrode 6.

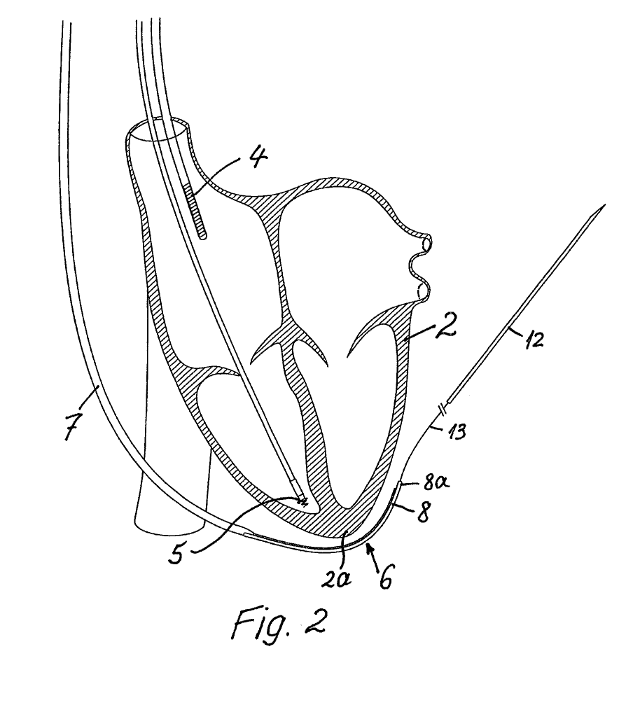

It is clear, above all in FIG. 2, that here the defibrillation electrode 6 is preferably flexible for encompassing the outside of the heart in the region of the cardiac apex 2a, in order to be able to at least partially take into account the curvature of the cardiac apex.

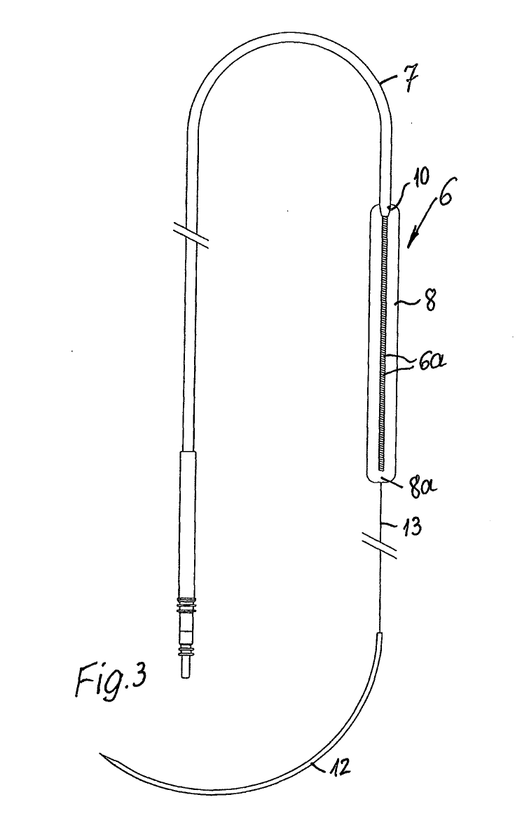

For this purpose, the defibrillation electrode 6 according to FIGS. 3 to 6 is a flexible helix made from metal or biocompatible steel with at least one helical-wound wire 6a or also two or more parallel-wound wires 6a, which promotes the desired bending capability and flexibility.

For all of the figures it follows that tensioning means or thread 13 is attached in a way still to be described to the defibrillation electrode 6 formed as a helix for its subcutaneous implantation, wherein the attachment of the tensioning element or thread to the defibrillation electrode 6 is formed so that the helix forming this electrode remains undilated during implantation and when a tensile force acts on the tension element or the thread 13, that is, its helical form that can be seen in FIGS. 3 to 6 remains unchanged to a large extent. The helix can and should produce just the curvature according to FIGS. 1 and 2 during implantation.

In order to keep the helix and thus the defibrillation electrode 6 free from the forces originating from the tension element or thread 13, according to all of the figures in the embodiments, this tension element or thread 13 attaches to a carrier 8 holding the helix, wherein this carrier 8 absorbs the tensile force during the implantation procedure and thus keeps it away from the helix 6. Here it can be seen that the tension element or the thread 13 is mounted on the front end region or end 8a of the carrier 8 in the insertion direction.

In all of the embodiments, a practically continuous helix is provided as a defibrillation electrode 6. This, however, could also be formed in the axial direction from several helical sections that are then connected to each other by wire pieces, in order to allow higher flexibility under some circumstances.

The feed line 7 that can be easily seen in FIGS. 1 to 4 to the helical defibrillation electrode 6 can here be an insulated low-impedance braid or also a helix. The insulation 10 is here easy to see in FIGS. 3 to 6 in that the helix forming the defibrillation electrode is an insulation-stripped projection of the helical feed line 7 located within the insulation 10 to the defibrillation electrode 6, which promotes the production of the entire arrangement. Here, within the wire forming the feed line 7 and/or the helix 6, there can be a silver matrix or tantalum matrix increasing the electrical conductivity.

Primarily in the FIGS. 3 to 6 one can see that the carrier 8 holding the defibrillation electrode 6 exceeds the length of the electrode 6, so that this is held securely accordingly.

In FIG. 5 it is shown that the defibrillation electrode 6 can also be formed by at least two helixes that are connected by a wire branching point 11 to the feed line 9 and nevertheless can increase the effectiveness of the defibrillation electrode 6 with a narrow and space-saving configuration.

These two helixes forming the defibrillation electrode 6 are mounted on the common carrier 8 extending parallel to each other, so that the tension element or the thread 13 can attach to the end 8a of this carrier 8 projecting opposite the helixes in the way already described and tensile forces on the tension element 13 do not deform the helixes.

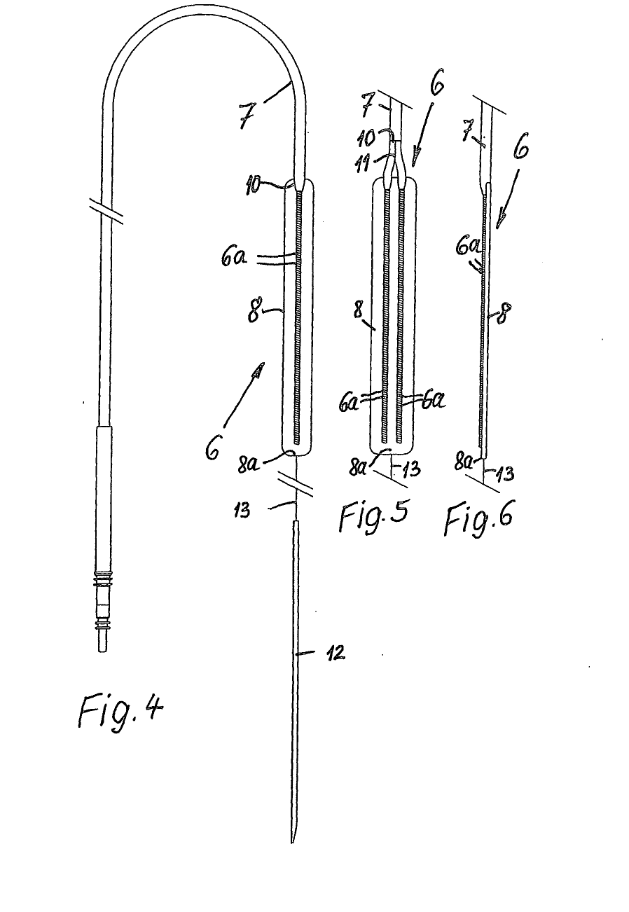

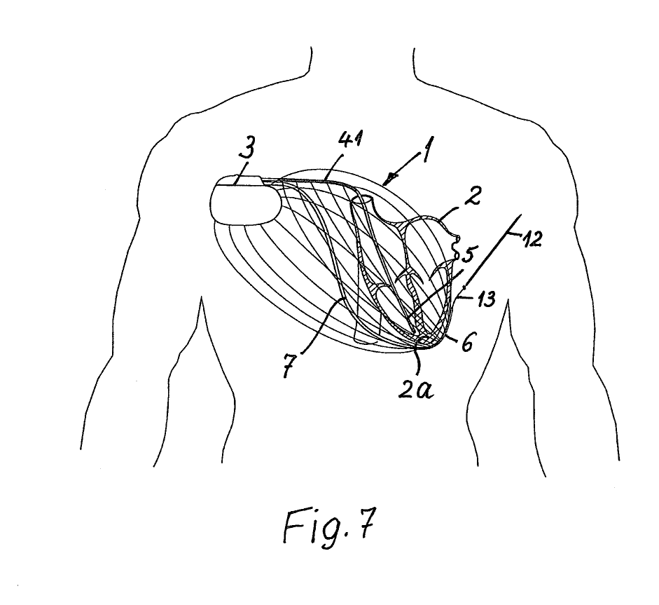

In FIG. 6, the helix or helixes are shown held by the carrier 8 on one side and also shielded opposite this side, so that this flat or approximately plate-shaped carrier 8 is arranged in the position of use according to FIGS. 1 and 2 on the side of the defibrillation electrode 6 facing away from the heart 2 and can be used for shielding. The defibrillation electrode 6 has a good action on the heart 2 accordingly.

The carrier 8 for this helical defibrillation electrode 6 here is formed preferably from insulating material and has, as insulating shielding according to FIGS. 3 to 5, a greater width than the defibrillation electrode 6 itself, even when this is formed from 2 helixes and projects opposite the defibrillation electrode 6 and also laterally opposite the helixes forming it, in order to form a correspondingly effective shielding. Due to the flat or plate-shaped formation and the selection of a correspondingly flexible material, the carrier 8 can also be easily curved and adapted to the anatomical conditions, thus it is flexible accordingly just like the helix or helixes.

The width of the carrier 8 acting as insulating shielding is here, for example, two-times or three-times or four-times as large as that of the defibrillation electrode 6 itself. The outer diameter of the helix or helixes forming the defibrillation electrode 6 can equal at least five-times, six-times, or seven-times the diameter of the wire forming the helix or helixes or can equal an intermediate value.

As a whole, an implantable device 1 is produced for the defibrillation of the heart, wherein the defibrillation electrode 6 of this device has improved reliability and, in particular, higher breakage resistance. Due to the highly flexible form with relatively small dimensions, wherein the diameter of the helix or helixes can equal approximately three-fourths to one millimeter, in particular, 0.8 to 0.9 millimeters, implantation of the defibrillation electrode 6 with its carrier 8 through a minimal, subcutaneous surgical intervention is possible in which the thread 13 can be drawn with the help of a needle 12 through the tissue close to the heart 2, after which the needle 12 that could be curved according to FIG. 3 or straight according to FIG. 4 is easily separated. The defibrillation electrode 5 is completely separated and the helical shape of also the feed line 7 produces a highly flexible and fracture-resistant feed line 7 with a long service life. The connection to the defibrillator 3 is realized with the help of the feed line 7 after it is drawn in with the help of the needle 12 underneath the heart 2 through the known tunneling method.

In FIGS. 7 to 9, an arrangement modified with respect to the counter electrode to the defibrillator electrode 6 is shown, wherein this counter electrode can be or is implanted outside of the heart 2.

In FIG. 7, a counter electrode 41 is shown that is arranged on the feed line of the stimulation electrode 5 outside of the heart on the outside of this feed line and leads with its own feed line to a plug in the cardiac pacemaker and defibrillator 3. Therefore, a good field-strength distribution and current flow through the heart 2 can be achieved, which is indicated schematically by corresponding field lines.

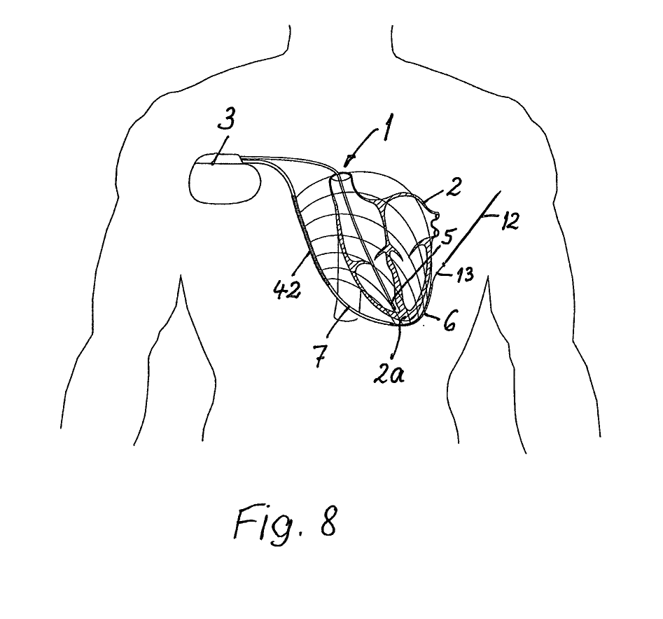

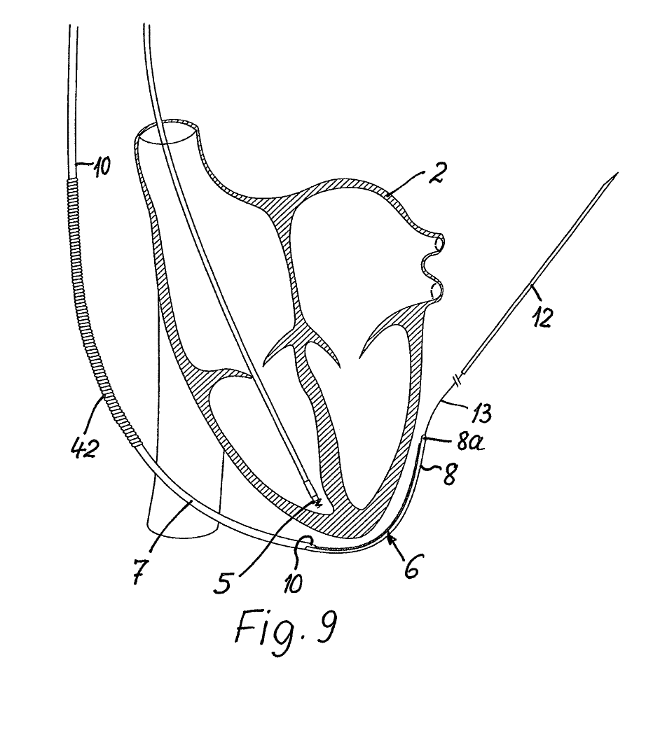

In contrast, FIG. 8 shows a modified embodiment in which the counter electrode 42 is also arranged outside of the heart 2 and is here arranged on the feed line 7 of the defibrillation electrode 6 on its outside, which leads to optimum field-strength distribution and optimum current flow through the heart 2 according to FIG. 8 and the shown field lines. Here, in FIG. 9 this arrangement is shown enlarged, so that one clearly sees the helical counter electrode 42 on the outside of the insulation 10 of the feed line 7, wherein the feed line to this counter electrode 42 cannot be seen in the drawing, just like the feed line to the counter electrode 41 in FIG. 7, because it is arranged within the insulation of the corresponding feed line, according to FIG. 9 within the insulation 10 of the feed line 7 of the defibrillation electrode 6, and runs from the counter electrode 42 or 41 to the cardiac pacemaker and defibrillator 3.

The device 1 is used for the defibrillation of the heart 2 and can be implanted as a whole. It features an implantable combination cardiac pacemaker and defibrillator 3, at least one defibrillation electrode 6, and a counter electrode 4, 41, or 42 for this defibrillation electrode, as well as a similarly implantable stimulation and sensing electrode 5, wherein the defibrillation electrode 6 can be drawn in and implanted subcutaneously close to the outside of the heart in the region of the cardiac apex 2a, for example, with the help of a tension element 13 and a needle 12 and is formed as at least one flexible helix made from metal or biocompatible steel, that is, has high flexibility and minimal space requirements.

1. Device (1) for the defibrillation of the heart (2) comprising an implantable, combination cardiac pacemaker and defibrillator (3), with at least one defibrillation electrode (6) and a counter electrode (4, 41, 42) for the defibrillation electrode, and also with at least one implantable stimulation and sensing electrode (5), wherein, in a position of use, the defibrillation electrode (6) is separated from the stimulation electrode (5) and is adapted to be implanted subcutaneously close to an outside of the heart in a region of the cardiac apex (2a), and the defibrillation electrode (6) is at least one flexible helix made from metal or biocompatible steel.

2. Device according to claim 1, wherein a tensioning element or thread (13) is provided on the defibrillation electrode (6) for its subcutaneous implantation, an attachment of the tension element or the thread to the defibrillation electrode (6) is formed so that the at least one flexible helix forming the defibrillation electrode remains undilated during implantation and when there is a tensile force acting on the tension element or the thread (13).

3. Device according to claim 2, wherein the tension element or the thread (13) used for the implantation of the defibrillation electrode (6) attaches at least to an end at a back in an insertion direction or end region of the at least one flexible helix or to a carrier (8) holding the helix, wherein the carrier (8) absorbs the tensile force during the implantation procedure and the force is kept away from the helix.

4. Device according to claim 3, wherein the tension element or the thread (13) attaches to or is mounted on a front end region or end (8a) of the carrier (8) in the insertion direction.

5. Device according to claim 1, wherein the at least one flexible helix used as the defibrillation electrode (6) is divided in an axial direction into several helix sections that are connected to each other by wires.

6. Device according to claim 1, wherein a feed line (7) to the defibrillation electrode (6) is an insulated, low-impedance braid or helix.

7. Device according to claim 6, wherein at least one flexible helix forming the defibrillation electrode (6) is an insulation-stripped projection of the feed line (7) to the defibrillation electrode (6).

8. Device according to claim 6, wherein within the wire forming the feed line (7) or helix (6), there is a silver matrix or tantalum matrix increasing an electrical conductivity.

9. Device according to claim 3, wherein the carrier (8) holding the defibrillation electrode (6) has approximately a same length of the electrode (6) or a somewhat greater length than the electrode (6).

10. Device according to claim 6, wherein the defibrillation electrode (6) is formed by at least two of the helixes that are connected to the feed line (7) by a wire branching point (11).

11. Device according to claim 10, wherein the two or more helixes forming the defibrillation electrode (6) are arranged or mounted on the common carrier (8).

12. Device according to claim 3, wherein the carrier (8) is flat or somewhat plate-shaped, and is arranged, in a position of use, on a side of the defibrillation electrode (6) facing away from the heart (2).

13. Device according to claim 3, wherein the carrier (8) for the defibrillation electrode (6) is made from insulating material and has, as insulating shielding, a greater width than the defibrillation electrode (6) itself and projects laterally past the helix or helixes forming them.

14. Device according to claim 13, wherein the width of the carrier (8) acting as the insulating shielding is two-times or three-times or four-times as large as a width of the defibrillation electrode (6).

15. Device according to claim 1, wherein an outer diameter of the at least one flexible helix forming the defibrillation electrode (6) equals at least five-times, six-times, or seven-times a diameter of the wire forming the at least one flexible helix or equals an intermediate value of three-fourths to one millimeter.

16. Device according to claim 1, wherein the counter electrode (4, 41, 42) for the defibrillation electrode (6) is formed as an atrial electrode that is adapted to be inserted transvenously into the heart (2) or can be implanted into the position of use outside of the heart (2).

17. Device according to claim 1, wherein the counter electrode (41, 42) is arranged outside of the heart (2) as at least one of a helix in the cardiac pacemaker, on a feed line (7) of the defibrillation electrode (6) or on the feed line of the stimulation electrode (5).

18. Device according to claim 1, wherein a feed line to the counter electrode (41, 42) is arranged within insulation of the feed line (7) of the defibrillation electrode (6) or the feed line of the stimulation electrode (5) and runs from the counter electrode to the cardiac pacemaker and defibrillator (3).
